(19) United States
(12) Patent Application Publication
(10) Pub. No.: US 2013/0085425 A1
(43) Pub. Date: Apr. 4, 2013

(54) METHODS AND SYSTEMS FOR VENTILATING OR COMPRESSING

(71) Applicants: Koenraad Monsieurs, Willebroek (BE); Alain Kalmar, Gent (BE)

(72) Inventors: Koenraad Monsieurs, Willebroek (BE); Alain Kalmar, Gent (BE)

(21) Appl. No.: 13/702,617

(22) PCT Filed: Jun. 9, 2011
(86) PCT No.: PCT/EP11/59592

§ 371 Date: Dec. 7, 2012

(30) Foreign Application Priority Data
Jun. 9, 2010 (GB) 1009666.7

(51) Int. Cl. A61H 31/00; A61M 16/00

(52) U.S. Cl. CPC A61H 31/004; A61M 16/00
USPC 601/41; 128/204.23

(57) ABSTRACT

A system for providing control signals for ventilating or compressing, respectively, includes an information receiving device that receives, for a resuscitation, information regarding a compression parameter and/or ventilation parameter, as function of a parameter indicative of blood circulation, a processing component for evaluating the different values of the chest compression parameter and/or ventilation parameter as function of the parameter indicative of blood circulation and deriving based on said information a value for the ventilation parameter and/or chest compression parameter respectively, and a control signal generator for generating control signals according to the derived ventilation parameter or chest compression parameter.

### FIELD OF THE INVENTION

The present invention relates to the field of medical devices. More particularly, the present invention relates to methods and systems for analysing resuscitation and to methods and systems for controlling ventilation and/or compression during resuscitation.

### BACKGROUND OF THE INVENTION

When a patient, such as a human being or an animal, needs positive pressure ventilation or chest compression (resuscitation), a number of clinical problems may arise.

One known clinical problem is the occurrence of increased intrathoracic pressures during resuscitation. There are numerous case reports of restoration of a spontaneous circulation after cessation of resuscitation efforts. This phenomenon, also referred to as the “Lazarus phenomenon” is mainly explained by trapping of air during ventilation and the presence of “positive end expiratory pressure” (PEEP) resulting in inefficacy or failure of the resuscitation. As trapped air escapes and the positive end expiratory pressure disappears after cessation of the resuscitation, this may allow blood to start flowing to the heart again and therefore result in restoration of circulation even after CPR efforts have been stopped.

Animal studies have also shown that hyperventilation during resuscitation results in decreased coronary perfusion pressure and in excess mortality. In a small clinical observational study of 13 patients with cardiac arrest, high ventilation rates and increased intrathoracic pressures were recorded. Hyperventilation is common during resuscitation. Such findings have resulted in the international recommendation to avoid hyperventilation during resuscitation for cardiac arrest.

Early detection and avoidance of hyperventilation and subsequent increased intrathoracic pressures during resuscitation may be an accurate means for preventing failure of resuscitation and for increasing survival chances and therefore is an important clinical issue.

Current state of the art methods to assess quality of resuscitation mainly use impedance measurement of the chest wall and accelerometers placed on the breastbone. The quality of ventilation is often currently addressed by impedance measurements between two electrodes attached to the chest of the victim. This provides reasonably accurate measurements of ventilation frequency and very rough measurements of volume. The quality of chest compression is determined by accelerometers placed on the breastbone of the victim. These provide reasonably accurate measurements of compression frequency and dept.

All these technical solutions to improve the quality and safety of intubation, ventilation and chest compression are in their early stages of clinical application and there is room for improvement.

### SUMMARY OF THE INVENTION

It is an object of embodiments of the present invention to provide good methods and systems for controlling ventilation and/or compression adapted to the requirements of the individual patient. In other words, methods and systems for ventilating and or compressing which take into account the particularities of the person or animal requiring it can be obtained.

It is an advantage of embodiments according to the present invention that an individualized resuscitation method can be obtained, individualized an optimized for the individual patient treated at that moment. Embodiments of the present invention allow determining the cardiac and thoracic pump potential during resuscitation in individual patients, thus also allowing individual, patient-dependent, optimization. It is an advantage of embodiments according to the present invention that the cardiac output of individual patients can be optimized.

It is an advantage of embodiments according to the present invention that methods and systems for ventilation and/or compression can be provided whereby control signals allow for improved ventilation and/or compression. It is an advantage of embodiments according to the present invention that during resuscitation, compression depth and ventilation strategies can be tailored.

It is an advantage of embodiments according to the present invention that efficient and accurate automated and automatic ventilation and/or compression systems can be obtained.

It is an advantage of embodiments according to the present invention that anatomical and physiological differences between patients can be taken into account as values of individual measurements are used for optimizing the ventilation and compression specifically for the individual patient.

The above objective is accomplished by a method and device according to the present invention.

The present invention relates to a system for providing control signals for ventilating or compressing, respectively, the system comprising an information receiving means for receiving information of a resuscitation for an individual patient, the information being information regarding different values of a chest compression parameter and/or ventilation parameter as function of a parameter indicative of blood circulation, a processing component for evaluating the different values of the chest compression parameter and/or ventilation parameter as function of the parameter indicative of blood circulation and deriving based thereon a preferred value for the ventilation parameter and/or chest compression parameter, and a control signal generator for generating control signals according to the derived preferred value of the ventilation parameter and/or chest compression parameter respectively. It is an advantage of embodiments according to the present invention that a more efficient resuscitation can be provided. The information also may comprise information regarding a chest compression parameter and/or ventilation parameter as function of a tracheal pressure difference by chest compression. The latter may be a parameter indicative of blood circulation. It has been surprisingly found that the pressure differences occurring upon chest compression or blood circulation show an optimum for a given ventilation volume, so that for smaller ventilation volumes, the pressure difference by chest compression are lower. In some cases also for larger ventilation volumes, the pressure differences by chest compressions are lower. It is believed that with a good or high pressure difference a good forward blood flow can be induced. The information receiving means may be an information receiving means for receiving ventilation volume as function of a parameter indicative of blood circulation. The processing component may be adapted for evaluating the different values of a ventilation parameter as function of a parameter indicative of blood circulation.

The information receiving means may be adapted for providing different values of a compression and/or ventilation parameter corresponding with a range of ventilation volumes.

It is an advantage of embodiments according to the present invention that a quick determination of the optimal ventilation conditions for obtaining optimum pressure difference occurring upon chest compression or for obtaining good or optimum blood circulation can be performed, especially as erroneous resuscitation induces higher risks for the patient. It is an advantage of embodiments according to the present invention that a quick determination of the optimal ventilation conditions for obtaining an optimum thoracic pump can be performed. It is an advantage of embodiments according to the present invention that a ventilator or compressor can be automated.

The information receiving means may comprise a pressure sensor for sensing tracheal pressure.

It is an advantage of embodiments according to the present invention that the compression related parameter can be determined based on tracheal pressure sensing.

The information receiving means or the processing component may comprise a calculator for calculating a parameter representative for the pressure difference by chest compression and/or a ventilation pressure or volume setting respectively based on tracheal pressure values.

It is an advantage of embodiments of the present invention that measurement of tracheal pressure, distal and/or proximal, may allow for determining the required information for obtaining accurate resuscitation.

The information receiving means, the processing means and the signal control generator may be part of a feedback loop, the system being adapted for, starting from a given ventilation volume/pressure or pressure difference by chest compression respectively, providing a control signal corresponding to another parameter value for a ventilation volume/pressure or a stronger/deeper chest compression,
receiving information regarding a parameter representative for the ventilation and/or compression as function of a parameter indicative of blood circulation, evaluating the ventilation parameter value and/or compression parameter value as function of the compression parameter indicative of blood circulation, and repeating said providing, receiving and evaluating until a parameter value indicative of a predetermined level or maximum level of blood circulation has been reached, e.g. a maximum pressure difference by chest compression has been reached.

It is an advantage of embodiments according to the present invention that an automated ventilator or compressor can be obtained whereby the optimum is found through a feedback loop, resulting in patient optimized conditions without the risk for applying too strong ventilation or compression.

The control signal generator may be adapted for selecting a control signal corresponding with the ventilation parameter value and/or the compression parameter value according to the predetermined level or maximum level of blood circulation.

It is an advantage of embodiments according to the present invention that selection of the optimum conditions can be performed.

The information receiving means may furthermore be adapted for obtaining end-tidal carbon dioxide measurements.

The system may furthermore comprise a ventilator or compressor respectively, the system thus being a ventilating system or compressing system.

The system may be implemented as a computer program product for, when executing on a computer, performing providing control signals for ventilating or compressing.

The present invention also relates to a method for providing control signals for ventilating or compressing, respectively, the method comprising receiving information of a resuscitation of an individual patient, the information being information regarding different values of a chest compression parameter and/or ventilation parameter as function of a parameter indicative of blood circulation, evaluating the different values of the chest compression parameter and/or the ventilation parameter as function of the parameter indicative of blood circulation and deriving based thereon a preferred value for the ventilation parameter and/or chest compression parameter, and generating control signals according to the derived preferred value of the ventilation parameter and/or chest compression parameter for controlling ventilation and/or compression. The method may comprise, starting from a given ventilation parameter or chest compression parameter, providing a control signal corresponding to a different ventilation parameter value or a different chest compression parameter value, receiving information regarding a chest compression parameter or ventilation parameter as function of a parameter indicative of blood circulation, evaluating the ventilation parameter value and/or compression parameter value as function of the pressure difference by chest compression or as function of blood circulation, and repeating said providing, receiving and evaluating until an maximum pressure difference by chest compression or good or optimum blood circulation has been reached. The maximum pressure may be an optimum pressure or a maximum pressure provided it does not strongly influence venous return. The present invention also relates to a data carrier comprising a set of instructions for, when executed on a computer, performing a method for providing control signals for ventilating or compressing, respectively, the method comprising receiving information of a resuscitation of an individual patient, the information being information regarding different values of a chest compression parameter and/or ventilation parameter as function of a parameter indicative of blood circulation, evaluating the different values of the chest compression parameter and/or the ventilation parameter as function of the parameter indicative of blood circulation, deriving based thereon a preferred value for the ventilation parameter and/or chest compression parameter, and generating control signals according to the derived ventilation parameter and/or chest compression parameter for controlling ventilation and/or compression.

The data carrier may be any of a CD-ROM, a DVD, a flexible disk or floppy disk, a tape, a memory chip, a processor or a computer.

Particular and preferred aspects of the invention are set out in the accompanying independent and dependent claims. Features from the dependent claims may be combined with features of the independent claims and with features of other dependent claims as appropriate and not merely as explicitly set out in the claims.

The teachings of the present invention permit the design of improved methods for ventilation and/or compression, more generally in improved methods for resuscitation.

The above and other characteristics, features and advantages of the present invention will become apparent from the following detailed description, taken in conjunction with the accompanying drawings, which illustrate, by way of example, the principles of the invention. This description is given for the sake of example only, without limiting the scope of the invention. The reference figures quoted below refer to the attached drawings.

### BRIEF DESCRIPTION OF THE DRAWINGS

FIG. 1 is a schematic representation of a system for analysing resuscitation according to an embodiment of the present invention.

FIG. 2 is a schematic representation of a flow chart of the algorithm that may be used for deriving information for the analysis of resuscitation according to an embodiment of the present invention.

FIG. 3 is a schematic representation of an exemplary tracheal ventilation pressure curve for oral intubation and mechanical ventilation as can be used in an embodiment according to the present invention.

FIGS. 4A and 4B are schematic representations of an exemplary tracheal ventilation pressure curve on the one hand (FIG. 4A) and an exemplary oesophageal ventilation pressure curve on the other hand (FIG. 4B), as can be used in embodiments according to the present invention.

FIG. 5a, FIG. 5b, FIG. 5c and FIG. 5d illustrate pressure curves for a distal measurement point and a proximal measurement point in case of tracheal intubation (FIG. 5a and FIG. 5b) and in case of oesophageal intubation (FIG. 5c and FIG. 5d) as can be obtained according to embodiments of the present invention.

FIG. 6 is a schematic representation of a computing device as can be used for performing processing steps in a method for analysing resuscitation according to an embodiment of the present invention.

FIG. 7 is a schematic flow chart illustrating an algorithm for determining a clinical relevant parameter, according to an embodiment of the present invention.

FIG. 8a, FIG. 8b and FIG. 8c illustrate output windows displaying the received pressure curves and derived clinical parameters according to an embodiment of the present invention (FIG. 8a) as well as output windows for insufflation analysis for a mechanical ventilation without CPR (FIG. 8b) and with CPR (FIG. 8c) as can be obtained according to embodiments of the present invention.

FIG. 9 illustrates a number of steps illustrating the functionality of at least part of a method for generating control signals for controlling a ventilator and/or compressor, according to an embodiment of the present invention.

FIG. 10 illustrates an exemplary system for providing control signals for a ventilator and/or compressor, according to the present invention.

FIG. 11 illustrates the variability of the pressure difference by chest compression for a plurality of patients, illustrating features and advantages of embodiments of the present invention.

FIG. 12 illustrates the initial difference in pressure by compression as function of the ventilatory pressure for a plurality of individuals, illustrating features and advantages of embodiments according to the present invention.

FIG. 13a to FIG. 13c illustrates a number of examples of individual measurements for the chest compression as function of the ventilatory pressure during resuscitation, as can be used in embodiments according to the present invention.

FIG. 14 illustrates the ventilation pressure for having the highest pressure difference by compression for a plurality of individuals, illustrating features and advantages of embodiments according to the present invention.

FIG. 15 illustrates deep and superficial pressure for three individual patients, illustrative of advantages of embodiments of the present invention.

FIG. 16a to FIG. 16e illustrate the effect of variation of different resuscitation parameters on the end-tidal CO₂ for an individual patient, illustrative of advantages of embodiments of the present invention.

FIG. 17 illustrates the pressure difference ΔCP and the deep measured pressure signal over time for an individual patient, illustrative of features and advantages of embodiments of the present invention.

The drawings are only schematic and are non-limiting. In the drawings, the size of some of the elements may be exaggerated and not drawn on scale for illustrative purposes.

Any reference signs in the claims shall not be construed as limiting the scope.

### DETAILED DESCRIPTION OF ILLUSTRATIVE EMBODIMENTS

The present invention will be described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes.

Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated.

Furthermore, the terms first, second, third and the like in the description and in the claims, are used for distinguishing between similar elements and not necessarily for describing a sequence, either temporally, spatially, in ranking or in any other manner. It is to be understood that the terms so used are interchangeable under appropriate circumstances and that the embodiments of the invention described herein are capable of operation in other sequences than described or illustrated herein.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention.

In the description provided herein, numerous specific details are set forth. However, it is understood that embodiments of the invention may be practised without these specific details. In other instances, well-known methods, structures and techniques have not been shown in detail in order not to obscure an understanding of this description.

Where in embodiments according to the present invention reference is made to ventilation volume, reference is made to the amount of air or gas that is provided by the ventilator during ventilation. The latter results in a pressure being built up, which in the present application may be referred to as ventilation pressure.

Where in embodiments according to the present invention reference is made to blood circulation, the latter may refer to blood flow and/or blood pressure and advantageously refers to the combination of blood flow and/or blood pressure.

In a first aspect, the present invention relates to a system for providing control signals for ventilation or compression respectively. The system thus may be suitable for controlling a ventilator or compressor or may comprise a ventilator or compressor for performing ventilating or compressing. According to embodiments of the present invention, the system comprises an information receiving means for receiving for a resuscitation of an individual patient and advantageously during such a resuscitation. Such information thereby is information regarding different values of at least one chest compression parameter and/or ventilation parameter as function of blood circulation, i.e. more particularly as function of a parameter indicative of blood circulation. At least one chest compression parameter and/or at least one ventilation parameter may for example be a ventilation volume, the depth of compression, etc. but also may be one or more settings of the ventilator and/or of the compressor resulting in such parameters. Resuscitation thereby typically may comprise external chest compression and invasive or non-invasive ventilation. The system furthermore comprises a processing component for evaluating the different values of the chest compression parameter and/or the ventilation parameter as function of the parameter indicative of blood circulation and deriving based thereon a preferred value for the ventilation parameter and/or the chest compression parameter. Such a preferred value may be a value for the ventilation parameter and/or the chest compression parameter for which good, better or best blood circulation is obtained. The value for the ventilation parameter and/or the chest compression parameter may be a value resulting in the highest pressure difference occurring upon chest compression or in the best blood circulation. So, the value for the ventilation parameter and/or chest compression may be optimal in view of pressure differences occurring upon chest compression. Alternatively also venous return could be taken into account and the value for the ventilation parameter and/or chest compression parameter may be a value resulting in the highest pressure difference occurring upon chest compression that still provides good venous return or that has no negative effect on the venous return. Furthermore, the system comprises a control signal generator for generating control signals for providing ventilation and/or compression according to the derived preferred ventilation parameter value and/or chest compression parameter value. The system and/or method may be part of or be used in combination with a ventilator or compressor, although embodiments of the present invention are not limited thereto and the system and/or method also may be used with a monitor, may provide control signals for a user of a ventilator or compressor, or may provide control signals to a user providing ventilation or compression to a patient and thus providing the functionality of a ventilator or compressor. A ventilator may for example be a mechanical ventilator as well as with a device for manual ventilation e.g. a self-inflating bag device, or even a user performing this function. A compressor may be a compressor as known in prior art, i.e. a mechanical system or a user performing this function. It is an advantage of embodiments according to the present invention that patient-individualised control signals can be generated for controlling a ventilator or compressor. Alternatively, control signals may be generated that can be used by a rescuer applying ventilation or compression to a patient e.g. by displaying the control signals or by translating them in a sound.

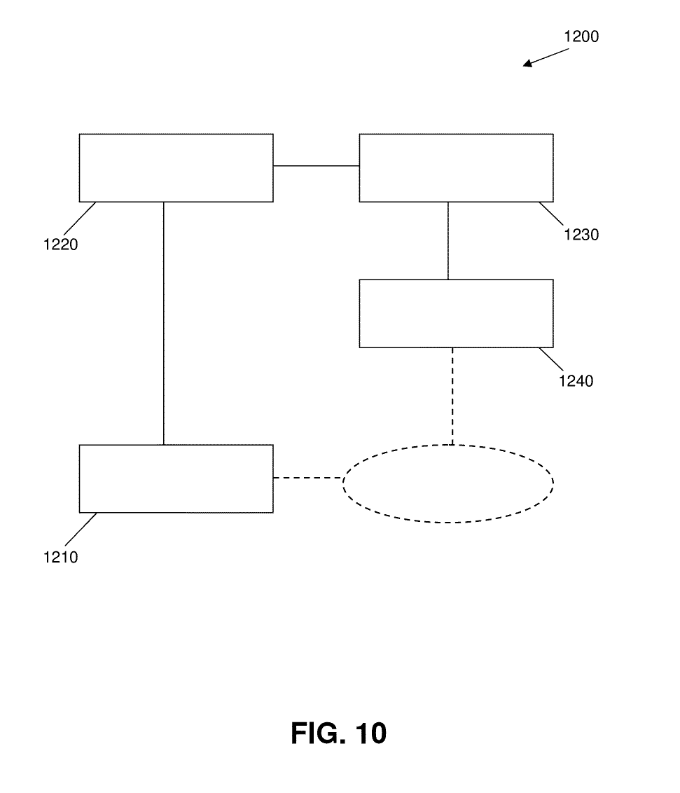

By way of illustration, embodiments of the present invention not being limited thereto, an example of as system for obtaining control signals is shown in FIG. 10. Embodiments of the system 1200 for providing control signals for controlling ventilating or compressing comprise an information receiving means 1210 for receiving information of a resuscitation of an individual patient, information regarding a compression parameter and/or ventilation parameter as function of a parameter indicative of blood flow. In other words, at least information regarding a parameter representative for the chest compression as function of a parameter indicative of blood flow may be received or at least a parameter representative for the ventilation as function of a parameter indicative of blood flow is obtained. This information may be prestored, precalculated, determined in the information receiving means 1210 itself, measured, etc. In one embodiment, the information receiving means 1210 is adapted with a tracheal pressure sensor for determining tracheal pressure for or during resuscitation. Such a tracheal pressure sensor may be adapted for determining plurality of tracheal pressure values over time for tracheal pressure during resuscitation. The number of tracheal pressure values advantageously is sufficiently high so that accurate details can be determined. In some embodiments, the tracheal pressure is sampled at a frequency of at least 1 Hz, more advantageously at least 10 Hz, still more advantageously at least 20 Hz, e.g. at least 50 Hz. According to some embodiments of the present invention, the information receiving means 1210 is adapted for receiving or obtaining measured tracheal pressure values for the patient. In some embodiments, the measured tracheal pressure values thereby advantageously are obtained at a distal end of the endotracheal tube, i.e. for example via a catheter inserted in the endotracheal tube intubated in the patient. Such signals may advantageously provide information regarding certain clinical parameters, not or less available in pressure signals captured at the proximal end of the endotracheal tube. Alternatively or in addition thereto, the measured values may be obtained further away from the distal end of the endotracheal tube, e.g. at the proximal end of the endotracheal tube. In some embodiments, measured tracheal pressure values may be obtained at at least two different positions in the endotracheal tube. The measured tracheal pressure values may for example be obtained at the distal end of the endotracheal tube and at the proximal end of the endotracheal tube. In some embodiments, combinations of such values may be used for deriving certain clinical parameters. In some embodiments, the measured tracheal pressure values may be measured when a supraglottic device is used or with a self-inflating bag device with a mask, i.e. some embodiments of the present invention relate to resuscitation without endotracheal tube. As described further below, receiving the measured tracheal pressure values may be receiving at an input channel of the system tracheal pressure values measured with a component not part of the system. Receiving measured tracheal pressure values then results in receiving corresponding data. When tracheal pressure is obtained as input in or via the information receiving means 1210, information regarding the compression and/or ventilation as function of a parameter indicative of blood circulation can be determined. The information regarding the compression and/or ventilation may be obtained by also capturing setting values of the ventilator and/or compressor or for example the corresponding ventilation volume or compression depth applied. One example of a way for determining the pressure difference by chest compression is by determining the pressure during chest compression and after or before chest compression. Determination of these pressures can be performed for example using techniques such as those described below. The information receiving means 1210 thus may receive or obtain information regarding a patient for a resuscitation or during a resuscitation. The information received may in some embodiments be information covering a range of values for the ventilation parameter and/or compression parameter.

The information received or obtained is further processed in a processing component 1220 for evaluating the different values of the chest compression parameter and/or the ventilation parameter as function of the parameter indicative of blood circulation and deriving based thereon a preferred value for a ventilation parameter and/or a chest compression parameter. The latter is based on the fact that it has surprisingly been found that the blood circulation or a parameter indicative thereof such as the pressure difference upon chest compression varies as function of the ventilation volume and the chest compression. The latter results in the fact that improving or optimization blood circulation, e.g. pressure difference occurring upon chest compression, can be performed by appropriately selecting ventilation parameter and/or compression parameter values. In advantageous embodiments at least the effect of ventilation on the pressure difference occurring upon chest compression or the blood flow is taken into account. In one embodiment, a maximum pressure difference by chest compression or good or optimum blood circulation can be found as function of the ventilation volume. In other words, by selecting the appropriate ventilation volume, an optimum pressure difference by chest compression or good or optimum blood circulation can be obtained, even without adjusting the chest compression. It is clear for the skilled person that, also for a given ventilation volume, selecting the chest compression parameter values, e.g. compression depth, also may result in a maximum pressure difference and thus methods and systems also are provided allowing such optimization. Furthermore, the present invention also relates to methods and systems whereby both ventilation pressure and chest compression are optimized for obtaining an optimum pressure difference by chest compression or good or optimum blood circulation. Determining values for a ventilation parameter and/or a compression parameter, e.g. by analysing the information received, may be performed using a predetermined algorithm, a neural network or according to predetermined rules. Ventilation parameter(s) and compression parameter(s) may be optimized subsequently or simultaneously.

As indicated compression also may be optimised for as function of blood circulation or a parameter indicative thereof. The compression parameter that may be used can be compression depth. The compression depth may for example be within a range between 4 cm and 6 cm for adult persons, e.g. within a range between 2 cm and 6 cm for children. In some embodiments the blood circulation can be measured through measurement of end-tidal CO₂, i.e. CO₂ in air outputted by a patient at the end of respiration. The latter is a measure for cardiac output during reanimation. In another embodiment, an algorithm for optimizing could alternately analyse and optimize compression and ventilator settings using a priority subalgorithm. This priority sub-algorithm can be based on a determination of minimal and maximal improvement potential of ΔCP of optimising compressor or ventilator settings respectively. For example, first the initial compression depth may be selected so that the compression depth corresponds with a conventional value chosen when applying conventional resuscitation. The ventilation settings can then be optimised using the initial compression depth, followed by subsequently optimising the compression settings for the obtained optimum ventilation settings. The algorithm can check whether the improvement is better than a predetermined value or relative value. If the improvement is better than a predetermined value, the algorithm decides that there is still room for improvement and a further optimisation cycle is performed. In one algorithm the optimisation may be performed by subsequently selecting the two best results out of three for the ventilation parameters or the compression parameters. In still further embodiments, the parameter indicative of blood circulation may be based on an image of the blood flow in a part of the body of the patient, such as for example of blood flow in the brain. Still another example may be measurement of the blood flow with an optical probe, e.g. positioned at a finger of the patient. The latter may measure blood flow by measuring a variation in the oxygen saturation curve. It is to be noticed that also other parameters can be used, in as far as they are directly or indirectly indicative of blood flow.

The system 1200 furthermore comprises a control signal generator 1230 for generating control signals for controlling the ventilator or compressor in agreement with the ventilation parameter value and/or chest compression parameter value derived with the processing component, or in other words, control signals for controlling ventilation or compression to be performed in agreement with the preferred ventilation parameter value and/or chest compression parameter value. Such control signals may be provided to a ventilator or compressor being part of the system, a ventilator or compressor not being part of the system both being controllable by electronic control signals, to a mechanical ventilator or compressor or even to a user performing ventilation or compression. The control signals thus may be electronic control signals, displayed control signals so as to be visible for a user, auditive control signals so as to be heard by a user, etc. The control signals may for example comprise whether or not more air is to be ventilated to the patient, more or less compression is to be provided, etc. In some embodiments optimization may be performed in a stepwise manner. For example, first one parameter may be optimized and thereafter, maintaining the first optimized parameter, further parameters may be optimized. For example, in one example first a value for the ventilation volume is determined resulting in high pressure difference occurring upon chest compression and thereafter, a ventilation frequency is optimized, in order to obtain a ventilation volume per minute. Optimisation of parameters may be performed within predetermined ranges, e.g. the value for the ventilation volume may be determined so that at least a minimum ventilation volume is provided. Such predetermined ranges may be defined by predetermined, e.g. clinically relevant, limit values. The starting value from where optimization may be performed may be a predetermined value, such as for example an agreed conventional value for the resuscitation parameter.

According to some embodiments, the system according to the present invention also comprises a ventilator or compressor 1240 for providing ventilation or compression to a patient or may cooperate therewith. Such a component may be part of the system 1200 or may be external thereto.

In some embodiments, one or more ventilation parameters and/or one or more compression parameters are optimized together. The latter can for example be obtained by providing a certain ventilation volume and blocking the airway temporary such that the air is kept in the thorax. The latter may for example be obtained using an inspiratory hold, whereby one valve is closed and air cannot escape from the thorax. During this phase, a compression parameter, e.g. compression depth, can be optimized. Further optimization can be performed in a next cycle where a different ventilation volume is used.

In some embodiments according to aspects of the present invention, a method is described for resuscitation, whereby a ventilation pressure is maintained in the thorax by blocking the airway temporary. By blocking the airway temporary for a couple of seconds and keeping the air in the thorax, compression can be performed for a particular ventilation volume, which may be selected so that optimal pressure difference is obtained for chest compression. The ventilation parameters and compression parameters may be determined using a method and/or system as described in aspects of the present invention. The present invention also relates to a controller for controlling a ventilator or compressor according to a method as described above and to a corresponding ventilator or compressor. In some embodiments both the ventilation parameters as well as a duration for blocking the airway may be set by the controller or may be implemented in the ventilation or compression system.

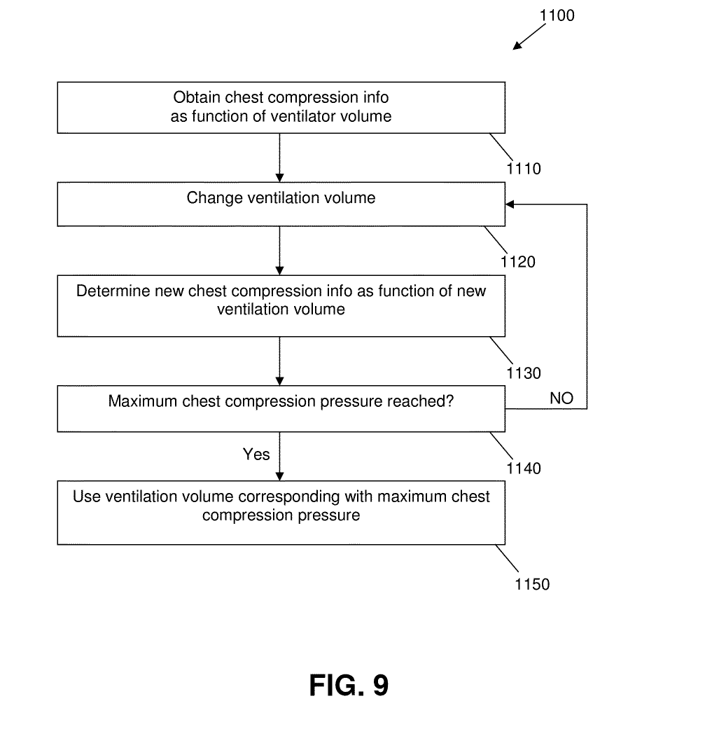

According to some embodiments of the present invention, the system is being adapted for providing feedback, e.g. with a feedback loop, whereby the information receiving means 1210, the processing component 1220 and the control signal generator 1230 are part of the feedback loop. In one embodiment, the system is adapted for building up information regarding a parameter representative for the compression or ventilation, as function of blood circulation, or a parameter indicative thereof, only for as far as required. The system may for example be programmed for performing steps as shown in FIG. 9, describing different method steps. The system may be adapted for obtaining initial blood circulation info as function of ventilation info, e.g. ventilation volume as shown in step 1110. Based on the ventilation volume used in the previous step, a new ventilation volume is obtained by incrementing the previous ventilation volume with a predetermined step, as indicated in step 1120 and by determining new blood circulation information with reference to the new ventilation volume, as indicated in step 1130. The blood circulation information is then compared with the blood circulation information obtained previously and it is determined whether a sufficient, good, optimum or maximum blood circulation is reached, as indicated in step 1140. If a sufficient, good or optimum blood circulation was reached in an earlier step, i.e. if a lower blood circulation is found, than the blood circulation is considered less than optimum, and the ventilation volume corresponding with the previously obtained best blood circulation is used for further ventilation 1150. If the best value for blood circulation was not reached yet, a new ventilation volume is determined by incrementing the ventilation volume, i.e. the system is programmed to return to step 1120. In a similar manner also optimization of compression may be determined for a fixed ventilation. For example, the blood circulation can be optimized as function of the compression depth, i.e. by selecting the appropriate compression depth, an optimal blood circulation can be obtained. In one embodiment, a method for detecting good values for a ventilation parameter such as for example ventilation volume is disclosed whereby sparse sampling is performed in a first step and whereby the new interval wherein sampling is performed is reduced in size each time by using the ventilation parameter values corresponding with the best pressure difference occurring upon chest compression or with the best blood circulation as edges of the new sampling interval. The latter results in a fast convergence.

Furthermore, the different algorithms may be repeated or continued over time, in order to deal with dynamic changes in the resuscitation process.

In accordance with some embodiments of the present invention a clinical parameter may be determined but this clinical parameter is not a diagnosis as such nor does it provide or lead to a diagnosis directly. That is, in accordance with some embodiments, the clinical parameter is only information from which relevantly trained personnel could obtain relevant medical conclusions however only after an intellectual exercise that involves judgement.

For determining the pressure difference by compression or the calculated ventilation volume, embodiments of the present invention may be adapted for analysing intrathoracic pressure during resuscitation. Other information obtained by analysis of intrathoracic pressure during resuscitation may also be used as further info to the user, e.g. rescuer. Alternatively or in addition thereto, the system may be adapted for providing an indication of a status of the patient or a status or quality of the resuscitation, i.e. provide an assessment of the patient or the resuscitation based on the obtained analysis results.

The information receiving means may make use of input from or may comprise one, more or all parts of a system for analysing tracheal pressure results. A corresponding system will be shown below, embodiments of the present invention not being limited thereto. Such a system for analysing tracheal pressure may be adapted for determining from said measured tracheal pressure values a tracheal pressure gradient. The tracheal pressure gradient may for example be a gradient of the measured tracheal pressure values, a gradient on smoothed tracheal pressure values or a gradient of the tracheal pressure values modified by subtracting an average tracheal pressure value determined in a moving window. The pressure gradient may be a temporal gradient of the measured tracheal pressure values, although embodiments of the present invention are not limited thereto and a spatial gradient of such pressure values also is envisaged. Such systems, and consequently also the information receiving means comprising such features, may be adapted for determining in real-time at least one clinical parameter based on the tracheal pressure values obtained. The clinical parameters may be a variety of clinical parameters such as for example the correctness of intubation including the location of the tube being intratracheal or oesophageal, or for example the quality of ventilation, including the occurrence of spontaneous ventilation and restoration of spontaneous circulation, i.e. spontaneous cardiac activity, the quality of obtained intrathoracic pressure, etc.

The system for analysing tracheal pressure data, which may be part of the system for providing control signals, may also be adapted for providing information regarding restoration of spontaneous ventilation and restoration of spontaneous circulation, i.e. spontaneous cardiac activity. In one embodiment, the system may be adapted for indicating whether a proper chest compression rate is achieved by the rescuer. In one embodiment, the system additionally may provide an indication of the ventilation frequency, e.g. including an indication or warning when the ventilation frequency is too high or too low. In another embodiment, the system may provide an indication of a wrong ventilation frequency and high pressures occurring. The system for providing control signals for controlling ventilation and/or compression as well as the system for analysing tracheal pressure data may be adapted in a hardware-based manner as well as in a software-based manner.

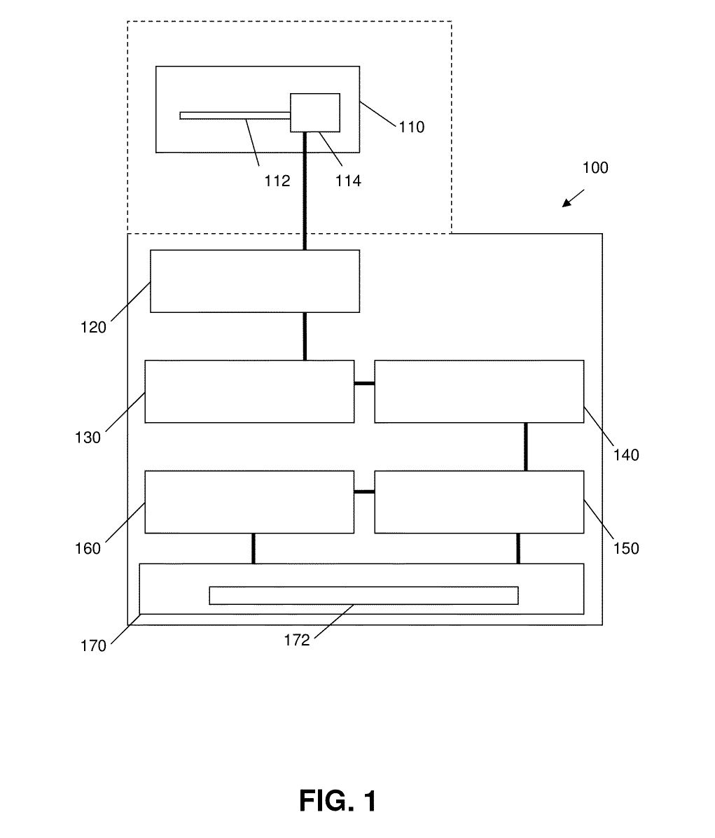

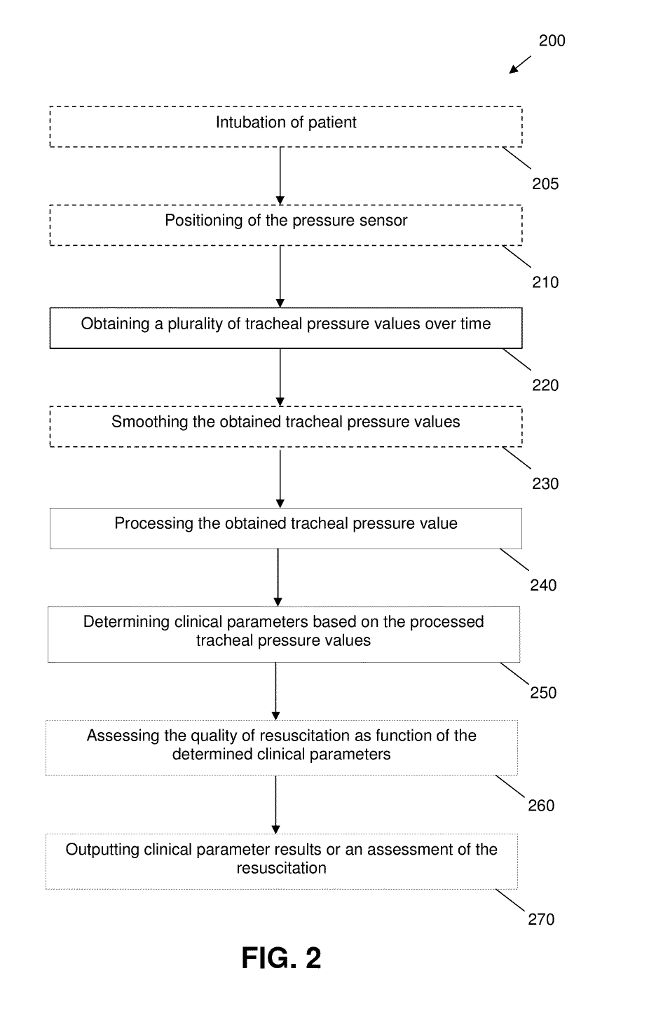

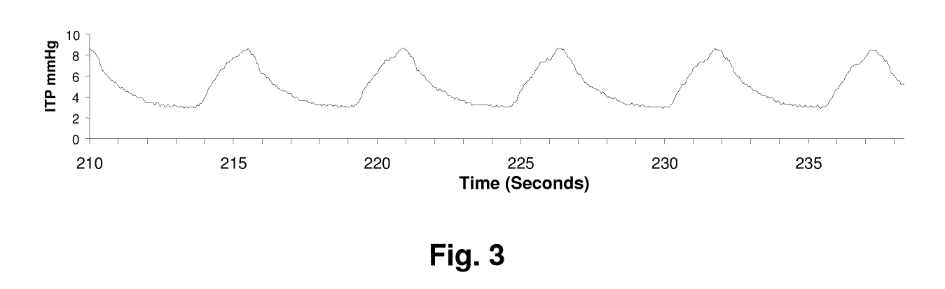

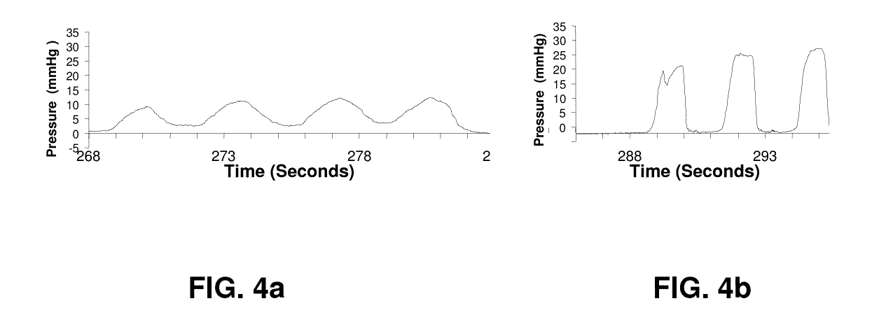

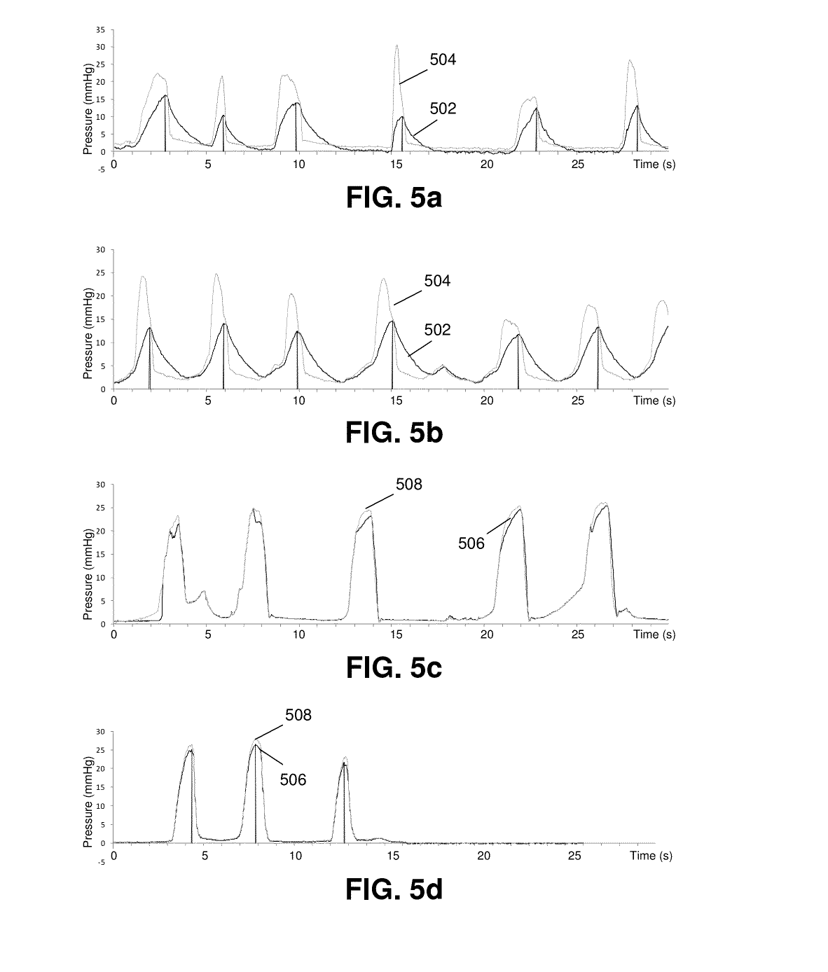

For the sake of completeness, embodiments of the present invention not being limited thereto, a description of an analysis system for analysing tracheal pressure as can be partly or fully part of the information receiving means is provided below. The exemplary system for analysing tracheal pressure is shown with reference to FIG. 1, indicating standard and optional components of a system for analysing resuscitation. The exemplary method is shown with reference to FIG. 2, indicating standard and optional steps of a method.

The system 100 may be provided with at least one pressure sensor 110 or it may be adapted to receive information from at least one pressure sensor 110. The at least one pressure sensor 110 may be any suitable pressure sensor for measuring pressure, advantageously a pressure sensor for measuring pressure at the distal end of the endotracheal tube. Alternatively or in addition thereto, a pressure sensor 110 also may be adapted for measuring pressure e.g. when using a supraglottic device or a self-inflating bag device with mask.

The at least one pressure sensor may be adapted for positioning a sensing part at the distal end of the endotracheal tube, e.g. close to the distal end of the endotracheal tube such as e.g. at about 2 cm from the distal end of the endotracheal tube of the patient. Alternatively, the at least one pressure sensor may be adapted for positioning a sensing part at the proximal end of the endotracheal tube. In some embodiments, tracheal pressure values may be determined at at least two different positions in the endotracheal tube. The latter provides the advantage that a spatial tracheal pressure gradient value can be determined, which may allow determination of clinical parameters in an accurate way. The at least one pressure sensor may be adapted for being inserted in the tube used when intubating the patient. One example of pressure sensor 110 that can be used is a catheter pressure sensor. The proximal end of such a catheter may optionally be connected to a bacterial filter and may be further connected to a pressure transducer. The catheter pressure sensor may comprise an air filled catheter 112, allowing to detect small variations in pressure. Pressure may be measured by transfer of a pressure signal sensed in catheter 112 to a pressure transducer 114, allowing to transfer the sensed signal into data. If detected in an analogue mode, the pressure data may be digitized. The pressure signal may, if appropriate intubation is performed, be a tracheal pressure signal. The obtained signal then is the sum of the pressure generated by positive pressure ventilation, chest compression, spontaneous breathing and spontaneous cardiac activity. The corresponding method 200 may optionally be adapted for measuring or assessing tracheal pressure signals using a pressure sensor as described above. The method thus may comprise intubating 205 the patient with an endotracheal tube and positioning 210 a pressure sensor for sensing intratracheal pressure or alternatively, it may be limited to a method initiated by obtaining pressure sensor data.

The system 100 and/or method 200 may be adapted for receiving or obtaining 220 measured tracheal pressure values. These samples may be received over any suitable telecommunications channel. For example, these values may be obtained via a wireless or a wired communication channel. The measured tracheal pressure values may be representative for a plurality of samples of the pressure over time. Advantageously, the sampling rate may for example be at least of at least 1 Hz, more advantageously at least 10 Hz, still more advantageously at least 20 Hz, e.g. at least 50 Hz. The latter results in a number of pressure values Pₓ at sampling points x, representative of time. The measured tracheal pressure values may be digitized or may be received in digitized form. The system may comprise an input means 120, also referred to as input port, for obtaining a plurality of tracheal pressure values over time. The input means 120 thereby may be adapted for receiving the pressure data directly from the pressure sensor 110 by performing the measurement act, whereby the system does not need to include the measurement equipment but only needs to be adapted for receiving the tracheal pressure data. Similarly, the method does not need to include the measurement act but only needs to be adapted for receiving as data input the tracheal pressure data.

The system 100 and/or method 200 furthermore is adapted for processing the obtained measured tracheal pressure values. Processing may include amplifying the signals using a suitable amplifier, such as for e.g. a Wheatstone Bridge amplifier. Advantageously, amplification is performed for each channel where tracheal pressure values are obtained. The amplifiers may be selected such that the range of amplification corresponds with the range of measured values, e.g. between −100 mbar and 100 mbar. The system 100 therefore may be adapted in hardware or in software. The system 100 may for example be equipped with processing capacity for performing the processing and may be programmed for performing the processing according to a predetermined algorithm, using a neural network or according to predetermined rules. The system 100 may be adapted for performing the receipt and the processing of the measured tracheal pressure values in an automated and/or automatic way. The processing may be performed in one or more central processors or may be performed in dedicated processing components. In the following description different components for performing the different processing steps will be indicated, but it will be clear to the person skilled in the art that the processing may be performed by the same processor. The processing tasks may be controlled by different software instructions, e.g. different steps in an algorithm. Similarly, intermediate as well as end results may be stored in one or a plurality of memories. Although in the following a single memory is described for storing intermediate and final results, the latter may be split up into several memories. The processing may be performed using a predetermined algorithm, allowing decomposition of the measured pressure signal in the individual contributions. Embodiments of the present invention are adapted for determining in real time at least one clinical parameter based on processing the obtained tracheal pressure values. The processing of tracheal pressure values may allow assisting in clinical assessment during resuscitation. As soon as a cycle of ventilation and/or compression has taken place, the clinical parameters can be determined substantially in real-time.

In a first optional processing step, smoothing 230 of the obtained measured tracheal pressure values may be performed. The system thus may be adapted for smoothing 230 the obtained measured tracheal pressure values, e.g. it may comprise a smoothing component 130 for smoothing. The smoothing component 130 may be software-based or may be dedicated hardware or a combination of software and hardware. The smoothing 230 may be performed to compensate for high frequency artefacts. Smoothing 230 may be performed by determining the mean pressure over a moving time-window of the measured pressure values and determining a smoothed tracheal pressure value there from. In one example, the time-window over which such averaging may be performed may be 150 milliseconds. In this way, the sampled tracheal pressure values may be transformed in a set of new smoothed tracheal pressure values by replacing every sampled value by its average in a time-window surrounding the sampled value. The latter may for example be obtained according to following algorithm, i.e.

For a number z of samples Pₓ

P₁, P₂, . . . P_{z}

the corresponding smoothed tracheal pressure value Sₓ can be determined by

$${S}_{x} = \frac{{\sum }_{i = - n + 1}^{0} {P}_{( x + i )}}{n}$$

wherein n is the number of samples in the moving time-window. For the initial n samples, the number of samples used for the smoothing may be gradually increased from 1 to n, or the initial values may be discarded. This smoothed waveform may be used for subsequent calculation of one, more or all of the ventilatory parameters of interest. Alternatively the non-smoothed measured pressure values may be used for further processing.

In a further processing step, the tracheal pressure values may be processed 240. The processing may comprise determining at least one tracheal pressure gradient value. Determining at least one tracheal pressure gradient value may be based on the smoothed tracheal pressure values or based on the measured tracheal pressure values without smoothing. Other processing also may be performed as described below. The system thus may be adapted for processing the tracheal pressure values, it may e.g. comprise a tracheal pressure value processing component 140 for processing the tracheal pressure values. The tracheal pressure value processing component 140 may be a tracheal pressure gradient calculation component for determining a tracheal pressure gradient value. The gradient thereby may be a temporal or spatial gradient. The temporal gradient, which may be expressed as dP/dt, expresses a variation of the pressure over time, whereas the spatial gradient, which may be expressed as dP/ds, expresses a variation of the pressure between two different locations. The tracheal pressure processing component 140 may be software-based or may be dedicated hardware or a combination of software and hardware.

The tracheal pressure gradient may be a temporal tracheal pressure gradient and/or a spatial tracheal pressure gradient. The tracheal pressure gradient may be a temporal tracheal pressure gradient determined based on a derivative over time of the tracheal pressure values. The temporal gradient in tracheal pressure may be determined by determining a derivative of the pressure waveform constituted by the tracheal pressure values, optionally the smoothed tracheal pressure values. In one embodiment, the latter is performed by determining the gradient of the ventilatory pressure in a time window around the sample or smoothed sample. In one example, the time window over which determination of the gradient may be performed may be 150 milliseconds. For samples Pₓ or the smoothed sample Sₓ the gradient value Gₓ may be determined as

$${G}_{x} = ( {P}_{x} - {P}_{( x - n )} ) * \frac{R}{n}$$

respectively

$${G}_{x} = ( {S}_{x} - {S}_{( x - n )} ) * \frac{R}{n}$$

whereby R is the sampling rate, n is the number of samples in the time window. Gₓ thereby is expressed in pressure per time unit.

According to embodiments of the present invention, the method and/or system furthermore is adapted for determining 250 at least one clinical parameter based on at least a pressure gradient value. The system thus may be adapted for determining at least one clinical parameter based on at least a pressure gradient value and therefore may comprise a clinical parameter determination component 150. The clinical parameter determination component 150 may be software-based or may be dedicated hardware or a combination of software and hardware. As already indicated above a plurality of clinical parameters may be determined based on at least a pressure gradient value obtained in the previous step. By way of illustration, some examples are provided, the invention not being limited thereto.

In another example, the gradient G may be used for determining the onset and release of chest compressions. When the gradient is above a predetermined value, e.g. above a predetermined cut-off value, a true compression may be suspected. If a gradient with a negative value of at least a predetermined value is subsequently detected within 500 ms and the highest pressure value between both gradient values is above a predetermined value, a true compression may be confirmed. The highest pressure value may be referred to as peak pressure. The system may be adapted to use the time between the two or some of the last maximal pressure values for determining a rate of chest compression. The system may be adapted for providing a notification when the determined chest compression rate is too high or too low. The lowest pressure value Pₓ in the 250 ms after the minimal gradient value Gₓ is the minimal pressure. Ideally, to achieve optimal venous return and blood flow to the heart, this value should be zero or negative. The system may be adapted for providing a warning or alarm notification if the minimal pressure does not return to baseline. Evaluation may be performed during several subsequent compressions. The latter may for example occur when there is incomplete release of compression. The system also may be adapted for determining a mean pressure generated by a chest compression. The latter may be determined by

$${P}_{m} = \frac{{\sum }_{i = {T}_{1}}^{{T}_{2}} {P}_{( i )}}{{T}_{2} - {T}_{1} + 1}$$

with point T₁ and T₂ being the time point of maximal Gₓ values of the two last compressions. The system furthermore may be adapted for determining a difference between the Peak Pressure and the Minimal Pressure, referred to as ΔP. If the amplitude of ΔP is too low, a warning or alarm notification may be provided.

In another particular example, the system is adapted for detecting spontaneous circulation. Spontaneous circulation may be evaluated based on a pulse pressure PP determined as follows: With M₁ being the minimal pressure value in a time span of 200 ms before the positive gradient value is obtained and M₂ being the minimal value in a time span of 200 ms after the negative gradient value, the minimum pressure can be determined as

$${P}_{min} = \frac{{M}_{1} + {M}_{2}}{2}$$

The peak pressure Pₚₑₐₖ can be determined as the highest pressure value between the positive gradient and the negative gradient.

The pulse pressure PP then is defined as

PP=Pₚₑₐₖ−Pₘᵢₙ

If the pulse pressure is higher than a minimal predetermined value, spontaneous circulation may be confirmed. Advantageously, also a gradient higher than a minimum value and a positive gradient value followed by a negative gradient value of minimal absolute value within 200 ms are factors pointing to spontaneous circulation. The combination of the above three aspects (pulse pressure, gradient value and subsequent positive and negative gradient) may allow confirmation of spontaneous circulation.

The tracheal pressure gradient may be a spatial tracheal pressure gradient based on tracheal pressure values determined at different positions in the endotracheal tube. The behaviour of the tracheal pressure values at the different positions may allow to derive the origin of pressure built up. If for example an abrupt pressure pulse is measured at the distal end of the endotracheal tube and a smaller pressure pulse is measured at the proximal end of the endotracheal tube, the tracheal pressure signal is more likely representative of a chest compression. If for example a weaker pressure pulse is measured at the distal end than the pressure pulse measured at the proximal end of the endotracheal tube, the tracheal pressure signal is more likely representative of a ventilation.

The method and/or system may be adapted for also determining further clinical parameters. The system therefore may comprise a additional parameter determination component 180. The system and/or method may for example be adapted for determining the mean pressure Mₓ at sample point x by averaging the sampled pressure values or the smoothed values thereof over a large time window, e.g. over a time window of 5000 ms. In further embodiments, this value may be used for determining whether the sampled pressure value or the smoothed sampled pressure value is below or above the mean pressure and the inversion point, for determining the highest value H of the sampled pressure values or the smoothed sample pressure values and/or for determining the lowest value L of the sampled pressure values or the smoothed sampled pressure values. Both timing and value of the maximal and minimal ventilatory pressure can be derived. Evaluation of the sign of ((Pₓ or Sₓ)−Mₓ) may allow to determine whether the sampled or smoothed sampled pressure is below or above mean pressure. Determination when ((Pₓ or Sₓ)−Mₓ) equals zero may allow to determine inversion points. Calculation of the mean pressure may be performed continuously, using a moving window.

The system optionally may be adapted for diagnosing a ventilation cycle, with a true sign inversion, if the highest sampled, optionally smoothed, pressure value minus the lowest sampled, optionally smoothed, pressure value is larger than a predetermined value, e.g. larger than 5 cmH₂O.

The system optionally may be adapted for determining the ventilation frequency based on the time between two sub-sequent peak ventilatory pressures. In another embodiment, the system may be adapted for determining within every ventilation cycle, the fraction of the time during which the ventilatory pressure is higher than a certain value. The obtained fraction may be used as signalling function, e.g. when the fraction is higher than a certain value an alarm signal may be provided. In yet another embodiment, the system may be adapted for determining whether a minimal ventilatory pressure is higher than a certain value. The latter may be used as signalling function, e.g. when the minimal ventilatory pressure is higher than a certain value, an alarm signal may be provided. This would signify the presence of PEEP and a risk of decreased venous return and lower efficacy of the chest compressions. The system may be adapted for providing an alarm signal if the ventilation frequency is or is repeatedly higher or lower than a certain value. The system may be adapted to provide an alarm signal if the maximal ventilatory pressure is higher than a certain value. In one embodiment, the system may be adapted for providing a notification of spontaneous respiration if a negative ventilatory pressure below a certain value is detected.

In a further step, the method and/or system advantageously may be adapted for assessing 200 the quality of the resuscitation based on the determined clinical parameters. Such an assessment may be performed in an automated and/or automatic way and results may be outputted or it may be performed by the user based on outputted determined clinical parameter results. The system 100 may be adapted with an assessment component 160 for assessing the resuscitation based on the determined clinical parameter results. The assessment component 160 may be software-based or may be dedicated hardware or a combination of software and hardware.

The method and/or system therefore advantageously also may be adapted for optionally generating 270 an output representative of the assessment of at least one clinical parameter or a related, e.g. physical, condition or an assessment of the resuscitation. The system therefore may comprise an output generating means 170. The latter may for example be a printer, plotter, speaker, display, lighting system, etc. The output may allow the user, e.g. rescuer, to maintain, adjust or stop his action. The output may be generated in a plurality of ways, the invention not being limited thereby. It may be data outputted on a plotter, printer or screen, it may be data outputted as sound signal or voice signal, it may be data visualised by colour, e.g. via coloured lamps, etc. or a combination of these. The system may be equipped with a user interface 172 for example allowing the user to select output information that he requires.

In some embodiments, the pressure data and/or clinical parameters may be stored in a memory, e.g. a memory of the system. The data thus can be recalled and used for debriefing and/or post-intervention evaluation of the resuscitation. Such information can be used for educational purposes or as a report of the resuscitation for medico-legal purposes.

The generated output may have a signalling or warning function. An often used way of generating output, the invention not being limited hereto, is activating a green light if the clinical parameter and/or the corresponding status of the patient or of the resuscitation is acceptable and providing a red light and/or sound signal if the clinical parameter and/or the corresponding status of the patient or of the resuscitation is not acceptable. If the system is part of a monitor, ventilator or defibrillator, outputting of information also may be performed through a single output system used by other components of the monitor, ventilator or defibrillator.

In order to further improve the information obtained with the system, some embodiments of the present invention comprise a system as described above, whereby the system furthermore is adapted with a detector for other signals that may be assisting in assessing clinical parameters, such as for example detection of ECG signals, end-tidal CO₂ measurement, detection of oxygen saturation, impedance measurements, accelerometric assessment of heart compression, etc. Combining of ECG signals with intrathoracic pressure level information according to an embodiment of the present invention may provide more accurate information regarding spontaneous cardiac activity and spontaneous respiration and thus enhancing the quality of the information. Combining the signals may allow further optimisation of decomposition of intrathoracic pressure values in its components. For example, appearance of a peak in the intrathoracic pressure systematically following the R-wave on an ECG indicates a higher probability of there being a true spontaneous cardiac compression than conclusions drawn when the ECG-information is absent. One possible example of such a detection is given by averaging several loops of the cardiac cycle by using the R-wave as reference starting point of the cycle and then averaging the intrathoracic pressure. Random artefacts should disappear in the averaged signal, while a systematic peak in the intrathoracic pressure would become more evident. The combined signals also may be outputted. Combining of the obtained results with end-tidal CO₂ measurements may provide information on the efficacy of the resuscitation effort. End-tidal CO₂ measurements can provide additional information regarding the result of the resuscitation, For example, end-tidal CO₂ measurements could provide further information regarding the overall effect of the optimisation of the pressure difference occurring upon chest compression and thus include effects on the venous return obtained.

The system for providing control signals as well as a system for analysing may be incorporated in existing ventilators or monitors. It thereby is an advantage that the system may be provided in software, so that implementation of the system can be performed relatively easy by installing software on existing systems. The ventilators or monitors further should be provided with a pressure sensor, which can be easily integrated in existing ventilators or monitors The system may be part of a portable monitor, defibrillator and/or ventilator. Alternatively, the system may be a separate device comprising or connectable to a pressure sensor.

It is an advantage of embodiments according to the present invention that one or more of the following data can be obtained: percentage positive pressure over total CPR time, positive end expiratory pressure, detection of spontaneous breathing, detection of spontaneous cardiac activity, incomplete release of compression, quality of intubation, mean and peak ventilation pressure, artificial ventilation frequency, rate of chest compression, mean and peak pressures generated by chest compression, ventilation and chest compression pauses, change of rescuers (by detecting a sudden change in pressure pattern) etc, both lists not being limiting.

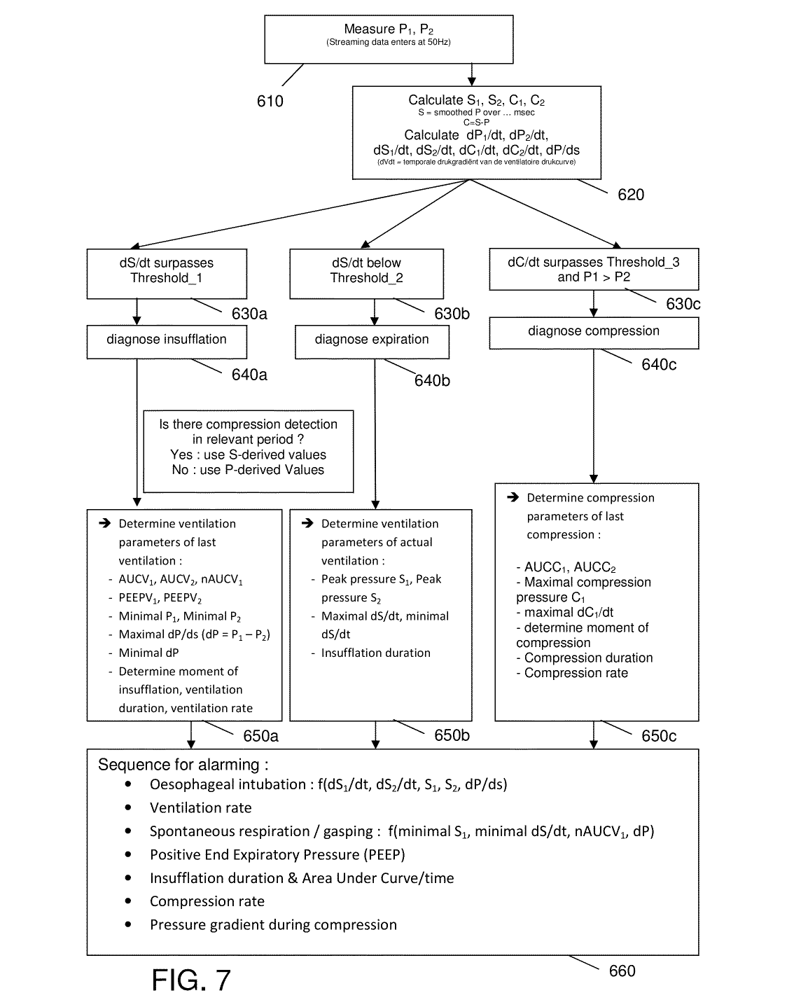

By way of illustration, embodiments of the present invention not being limited thereby, an example of an algorithm that may be used in a system or method as described in the first or second aspect, or in a processing system or computer program product as described in the third aspect, is illustrated in FIG. 7 by way of flow chart 600.

In a first step 610, measurement or receipt of tracheal pressure data is indicated. In the current exemplary algorithm, tracheal pressure values are obtained at two different positions, in this example illustrated by P₁ and P₂, embodiments of the present invention not being limited thereto, so measurements also could be performed at a single location or at more than 2 positions. In the present example P₁ expresses the pressure in the distal end of the endotracheal tube, i.e. used for sensing closer to the lungs, and P₂ expresses the pressure in the proximal end of the endotracheal tube, i.e. used for sensing further away from the lungs. Such values typically may be expressed in mbar. Measurement data typically may be obtained for different moments in time. The data typically may be obtained as streaming data, advantageously e.g. at a frequency sufficiently high to evaluate shape of the signal or the shape of a differential value thereof.

In a second step 620, at least a gradient based on the tracheal pressure value as function of time or position is determined. This may be one of the pressure gradients as described below. The number of parameters that can be calculated may be large. Advantageously following parameters can be calculated:
A ventilatory pressure value S based on the tracheal pressure, obtained by smoothing the tracheal pressure values obtained in a given time window. A series of data may be obtained by using a moving time window for the integration. S₁ and S₂ in the present example thus correspond with smoothed versions of P₁ and P₂ respectively. The smoothed values reflect the ventilatory pressure.
A compression pressure value C based on the tracheal pressure, obtained by subtracting the smoothed tracheal pressure value from the received tracheal pressure value, i.e. C=P−S, resulting in a modified tracheal pressure value reflecting the additional pressure generated by the compressions. In the present example modified pressure values C₁ and C₂ can be determined based on the received tracheal pressure values P₁ and P₂ respectively and on the smoothed tracheal pressure values S₁ and S₂ respectively.
A pressure gradient over time for the received tracheal pressure values, indicated as dP/dt. For the different tracheal pressure values, this can be indicated as dP₁/dt and dP₂/dt respectively.
A pressure gradient over time for the ventilatory pressure values S, indicated as dS/dt, indicating the pressure gradient over time of the ventilation pressure curve. For the different ventilatory pressure values, this can be indicated as dS₁/dt and dS₂/dt respectively.
A pressure gradient over time for the compression pressure values C, indicated as dC/dt, indicating the pressure gradient over time of the ventilation pressure curve. For the different compression pressure values, this can be indicated as dC₁/dt and dC₂/dt respectively.
A spatial pressure gradient, indicated as dP/ds, indicating the difference in pressure as function of position, e.g. the spatial pressure gradient between P₁ and P₂.

In a third step 630a, 630b, 630c, a clinical parameter is determined based on the processed tracheal pressure values. Different clinical parameters can be determined as illustrated by steps 630a, 630b and 630c.

In a first example in step 630a it is evaluated whether the pressure gradient over time of the ventilation pressure curve surpasses a given threshold, indicated as Threshold 1. Such a threshold may be a value suitable for detecting the start of insufflation. The derived clinical parameter thus is whether or not the gradient over time of the ventilation pressure surpasses a given threshold. Depending on the fulfilment of the condition a diagnosis of insufflation may be made through judgment of relevantly trained people, as indicated in step 640a. For deriving further information, in step 650a, the ventilation parameters of the last ventilation may be determined, such as for example the area under the ventilation curve of ventilation pressure S₁, indicated as AUCV₁ the area under the ventilation curve of the ventilation pressure S₂, indicated as AUCV₂, the area under the ventilation curve for a negative ventilation pressure S₁ reflecting the duration and amplitude of negative detection for detection of gasping and spontaneous breathing, indicated as nAUCV₁, the positive end-expiratory pressure of the ventilatory curve for ventilation pressure S₁ and S₂, indicated as PEEPV₁ and PEEPV₂ respectively, the minimal tracheal pressures for P₁ and P₂ being the lowest detected pressure within the ventilation cycle which can be used for detection of gasping, the maximal spatial pressure gradient dP/ds, whereby dP is given by the difference in tracheal pressure P₁−P₂, the minimal spatial difference in tracheal pressure, i.e. the minimum dP, the moment of insufflation, the ventilation duration, the ventilation rate, etc. dP/ds thereby relates to the flow (e.g. in ml/sec) and thus can be used to determine the volumes of displaced air, i.e. the breathing volume.

In a second example in step 630b, it is evaluated whether the pressure gradient over time is below a given threshold, indicated as Threshold 2. Such a threshold may be a value suitable for detection of expiration. The derived clinical parameter thus is whether or not the gradient over time of the ventilation pressure is below the given threshold 2. Depending on the fulfillment of the condition a diagnosis of expiration may be made through judgment of relevantly trained people, as indicated in step 640b. For deriving further information, in step 650b, the ventilation parameters of the actual ventilation may be determined, such as for example the peak pressure of the ventilation pressure S₁ and S₂ which is the highest detected pressure within the ventilation cycle, the maximal pressure gradient over time for the ventilation pressure, which may be used for detection of oesophageal intubation, the minimal pressure gradient over time for the ventilation pressure, the duration of the insufflation, which may be used for evaluation of the quality of ventilation, etc.

In a third example in step 630c, it is evaluated whether the pressure gradient over time for the compression pressure surpasses a given threshold value, indicated as Threshold 3. Such a threshold may be a value suitable for detection of compression. Furthermore it is evaluated if, combined with the previous condition, the condition is fulfilled that the endotracheal pressure closest to the lungs P₁ is larger than the endotracheal pressure further away from the lungs P₂. The derived clinical parameter thus is whether or not the pressure gradient over time for the compression pressure is larger than a predetermined value and that P₁ is larger than P₂. Depending on the fulfillment of these conditions, a diagnosis of compression may be made through judgment of relevantly trained people, as indicated in step 640c. For deriving further information in step 650c, the compression parameters of the last compression also may be determined, such as for example the area under the compression curve of compression pressure C₁, indicated as AUCC₁ the area under the compression curve of the compression pressure C₂, indicated as AUCC₂, the maximal compression pressure C₁, the maximal compressive pressure gradient dC₁/dt for the compressive pressure values based on the endotracheal pressure values closest to the lungs, the moment of compression, the compression duration, the compression rate, etc.

In case compression is detected, the steps 650a and 650b may be performed using the ventilation pressure steps, whereas in other cases, the endotracheal pressure values may be used.

In step 660, the required results are outputted. In order to prevent a too large amount of information to be provided to the user, only the most relevant information may be provided to the user. Outputting also may be already partially performed after step 640a, 640b, 640c. One possible order of indication may be outputting information regarding oesophageal intubation, which is a function of the ventilation pressure gradients, the ventilation pressure values and the spatial gradient of the endotracheal pressure, then regarding the ventilation rate, then regarding respiration and/or gasping, which is a function of the minimal ventilation pressure, the minimal ventilation pressure gradient, the negative area under the ventilation curve and the difference between the endotracheal pressures, then regarding positive end expiratory pressure, then regarding the insufflation duration and the area under the curve per time, then regarding the compression rate and then regarding the pressure gradient during compression. The amount of info displayed may be selectable. The algorithm illustrates different aspects that may be implemented in software or hardware in systems of the present invention.

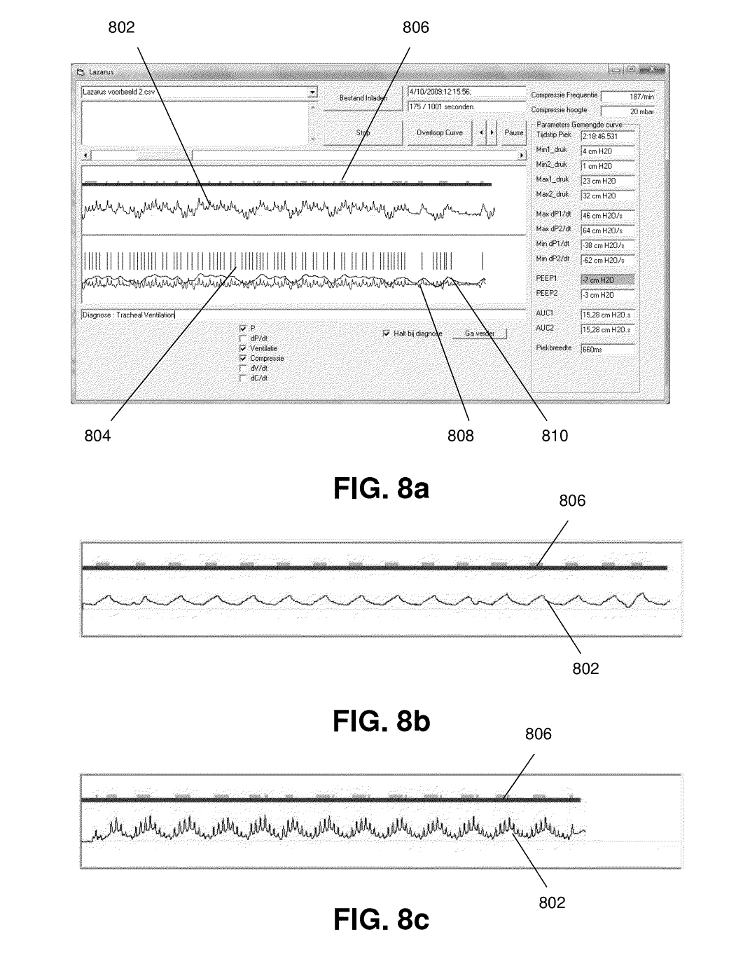

FIG. 8a, FIG. 8b and FIG. 8c illustrate an output window of software according to an embodiment of the present invention. In FIG. 8a, a recorded waveform 802 of CPR-pressure measurements is analysed. In the example shown, all relevant parameters are calculated in real time to determine physiological parameters. The thoracic compressions (stripes 804 in lower field) and insufflations (indicators 806 in upper field) are detected, the recorded waveform 802 is decomposed in a compression related pressure curve 808 and a ventilation related pressure curve 810. Analysis of the different parameters allows determination of the relevant physiological parameters. The system or associated software is adapted for informing the user if some of the parameters (see block diagram) are too different from the ideal values.

If multiple parameters are aberrant, a prioritizing algorithm is used to determine the most urgent and an alarm is given accordingly as was also discussed with reference to FIG. 7. FIG. 8b and FIG. 8c illustrate the insufflations (indicators 806) for both a mechanical ventilation without CPR and mechanical ventilation with CPR, as derived from the corresponding pressure curves 802.

Returning now to the concept of providing control signals for ventilating or compressing, in a second aspect, the present invention relates to a monitor, ventilator or defibrillator for providing resuscitation to a patient in need. The monitor, ventilator or defibrillator according to embodiments of the present invention comprises conventional components for allowing ventilation and/or defibrillation, but furthermore comprises a system for providing control signals for controlling ventilation and/or compression, as set out in the first aspect. The system may comprise the same features and advantages as set out above.

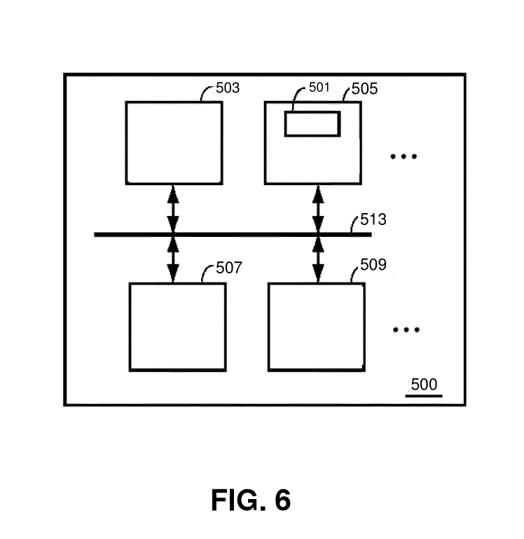

In a third aspect, the present invention relates to a processing system wherein the method or system for providing control signals for ventilating or controlling as described in embodiments of the previous aspects are implemented in a software based manner. FIG. 6 shows one configuration of a processing system 500 that includes at least one programmable processor 503 coupled to a memory subsystem 505 that includes at least one form of memory, e.g., RAM, ROM, and so forth. It is to be noted that the processor 503 or processors may be a general purpose, or a special purpose processor, and may be for inclusion in a device, e.g., a chip that has other components that perform other functions. Thus, one or more aspects of embodiments of the present invention can be implemented in digital electronic circuitry, or in computer hardware, firmware, software, or in combinations of them. The processing system may include a storage subsystem 507 that has at least one disk drive and/or CD-ROM drive and/or DVD drive. In some implementations, a display system, a keyboard, and a pointing device may be included as part of a user interface subsystem 509 to provide for a user to manually input information. Ports for inputting and outputting data also may be included. More elements such as network connections, interfaces to various devices, and so forth, may be included, but are not illustrated in FIG. 6. The various elements of the processing system 500 may be coupled in various ways, including via a bus subsystem 513 shown in FIG. 6 for simplicity as a single bus, but will be understood to those skilled in the art to include a system of at least one bus. The memory of the memory subsystem 505 may at some time hold part or all (in either case shown as 511) of a set of instructions that when executed on the processing system 500 implement the steps of the method embodiments described herein. Thus, while a processing system 500 such as shown in FIG. 6 is prior art, a system that includes the instructions to implement aspects of the methods for providing control signals and using them is not prior art, and therefore FIG. 6 is not labelled as prior art.

The present invention also includes a computer program product which provides the functionality of any of the methods according to the present invention when executed on a computing device. Such computer program product can be tangibly embodied in a carrier medium carrying machine-readable code for execution by a programmable processor. The present invention thus relates to a carrier medium carrying a computer program product that, when executed on computing means, provides instructions for executing any of the methods as described above. The term “carrier medium” refers to any medium that participates in providing instructions to a processor for execution. Such a medium may take many forms, including but not limited to, non-volatile media, and transmission media. Non volatile media includes, for example, optical or magnetic disks, such as a storage device which is part of mass storage. Common forms of computer readable media include, a CD-ROM, a DVD, a flexible disk or floppy disk, a tape, a memory chip or cartridge or any other medium from which a computer can read. Various forms of computer readable media may be involved in carrying one or more sequences of one or more instructions to a processor for execution. The computer program product can also be transmitted via a carrier wave in a network, such as a LAN, a WAN or the Internet. Transmission media can take the form of acoustic or light waves, such as those generated during radio wave and infrared data communications. Transmission media include coaxial cables, copper wire and fibre optics, including the wires that comprise a bus within a computer.

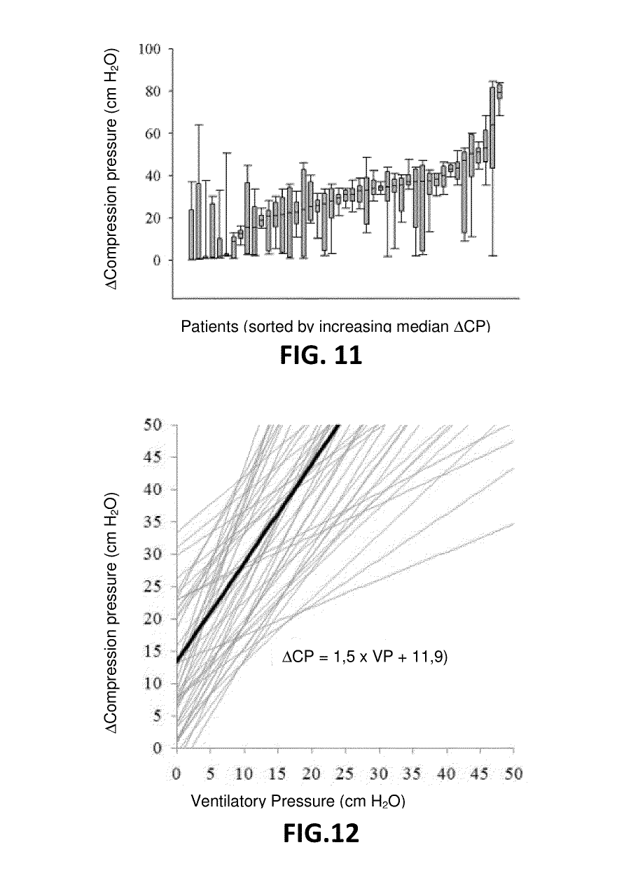

By way of illustration, embodiments of the present invention not being limited thereto, experimental results regarding cardiopulmonary resuscitation are discussed below. A study was performed whereby in 45 patients an out-of-hospital cardiopulmonary resuscitation was performed and airway pressure was measured at the proximal end of the endotracheal tube. The sampling frequency was at least 20 Hz, i.e. for some patients 20 Hz, for some patients 50 Hz. Using the first 60 seconds of the pressure waveform (either during manual or mechanical ventilation), The pressure difference by chest compression ΔCP was determined for each chest compression and the ventilation pressure VP at the time of compression was calculated. The pressure difference by chest compression ΔCP is a parameter indicative of the blood circulation. A high pressure difference may allow for a good blood circulation. Statistical analysis was performed to explore the relationship between pressure difference by chest compression ΔCP and ventilation pressure VP. FIG. 11 indicates the variability in pressure difference by chest compression ΔCP within and between individuals. Individual patients are sorted by increasing median for the pressured difference by chest compression ΔCP. For each patient, the median, 25th and 75th percentile (box) and the 10th and 90th percentile (whiskers) of the recorded ΔCP's are shown. The pressure difference by chest compression ΔCP ranged from 0 cm H2O to 82 cm H2O. The median value for pressure difference by chest compression ΔCP was 31 cm H₂O. Initially a positive correlation between pressure difference by chest compression and ventilation pressure was found. When ventilation pressure initially increased from 0 to 15 cm H₂O, the pressure difference at chest compression ΔCP was almost 4 times amplified. The latter can be seen in FIG. 12 correlating the initial pressure difference by chest compression and ventilation pressure.

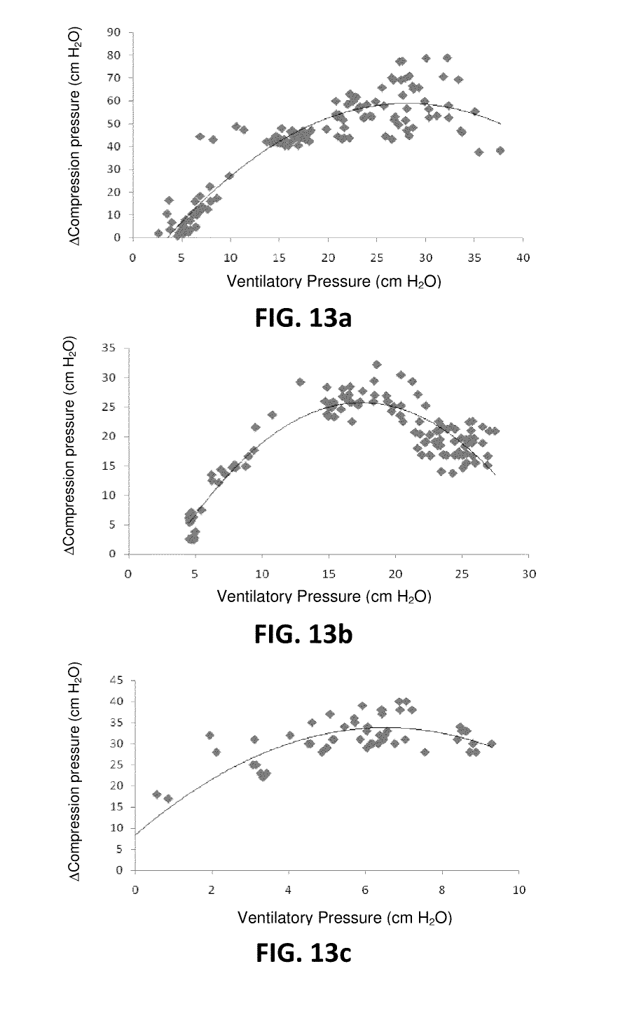

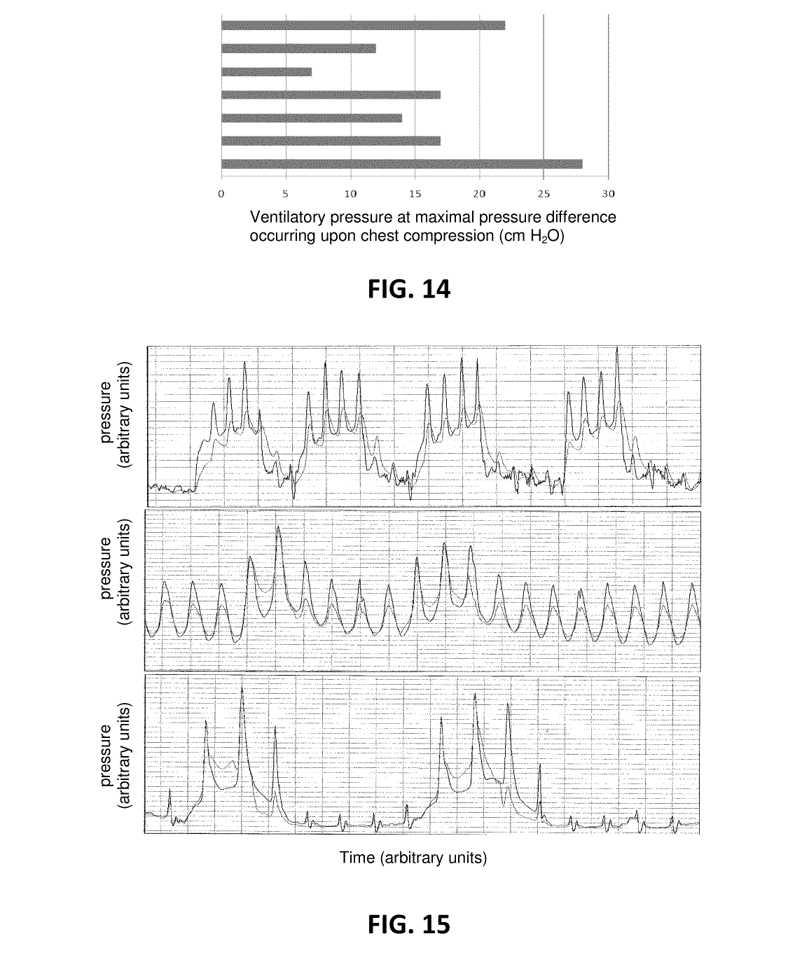

When the pressure difference by chest compression is evaluated for higher ventilation pressure, it can be seen that a maximum pressure difference for chest compression can be obtained for a given ventilation pressure. By way of example a number of measurements of individual resuscitations is shown in FIG. 13a to FIG. 13c. In these drawings, it can be seen that indeed an optimum can be reached as function of the ventilation pressure. Furthermore, it can be seen that for different patients, a different optimum ventilation pressure can be found. The maximum for seven different resuscitations is shown in FIG. 14.

Forward blood flow during cardiopulmonary resuscitation (CPR) is believed to be the result of direct compression of the heart (the “cardiac pump”) and intrathoracic pressure (ITP) differences (the “thoracic pump”). The ITP during CPR is a combination of pressure generated by ventilation (VP) and pressure differences generated by chest compression (ΔCP). The above results indicate not only that the chest compression can be optimized by selecting a ventilation pressure, but also that for different patients different resuscitation conditions should be applied, as the pressure difference generated by chest compression vary greatly within and between patients. By way of illustration, embodiments of the present invention not being limited thereby, examples of deep and superficial pressure signals for different patients are described in FIG. 15. The latter indicates that obtained pressure profiles for individual patients can differ significantly. The obtained pressure profile for the individual patient may depend on the age, gender, stiffness of bodily parts, etc. The latter illustrates that consequently also the optimum conditions for resuscitation of individual patients differ significantly, as can be taken into account using embodiments of the present invention.

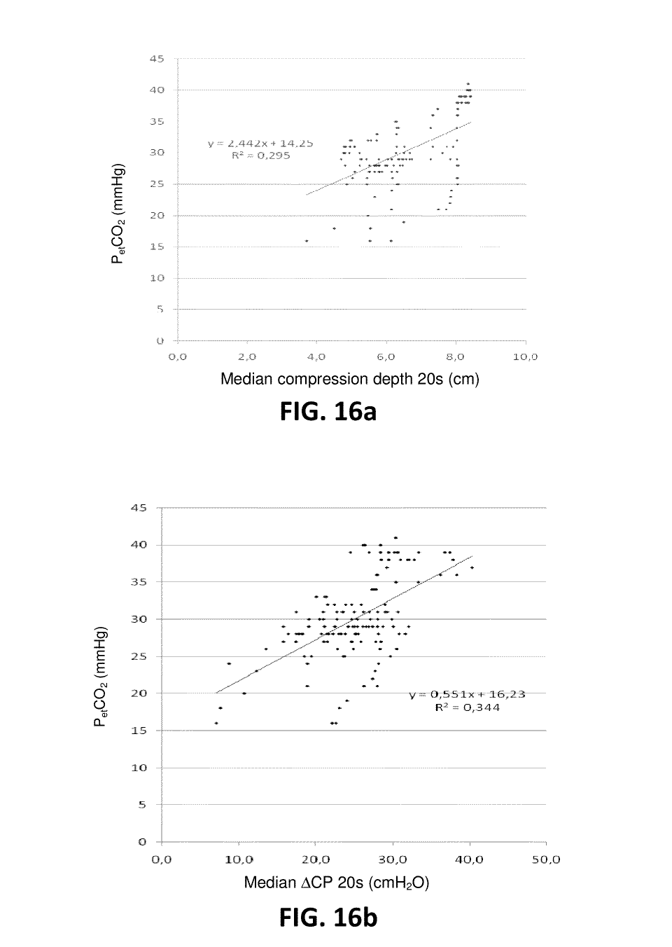

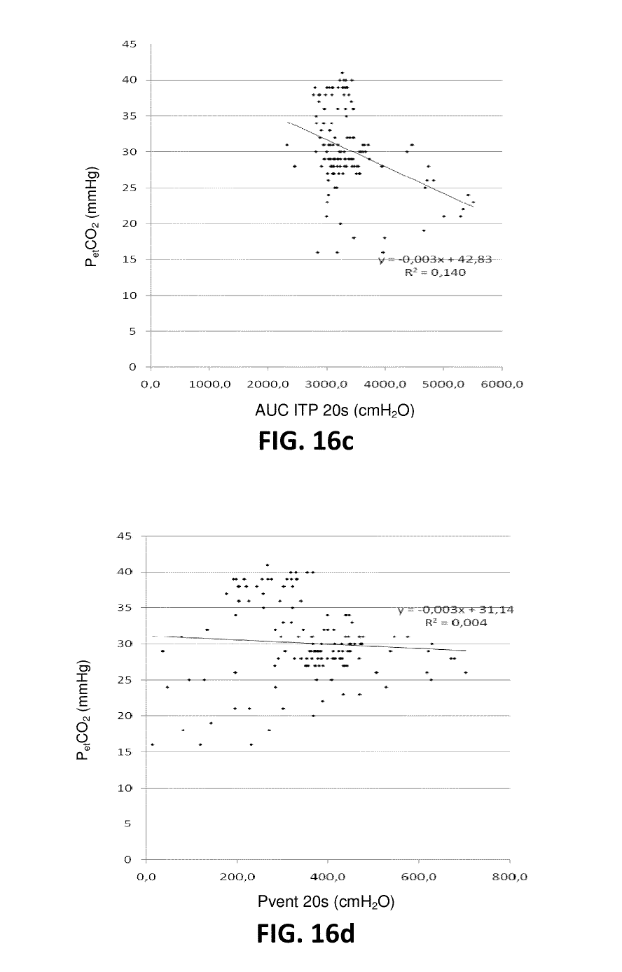

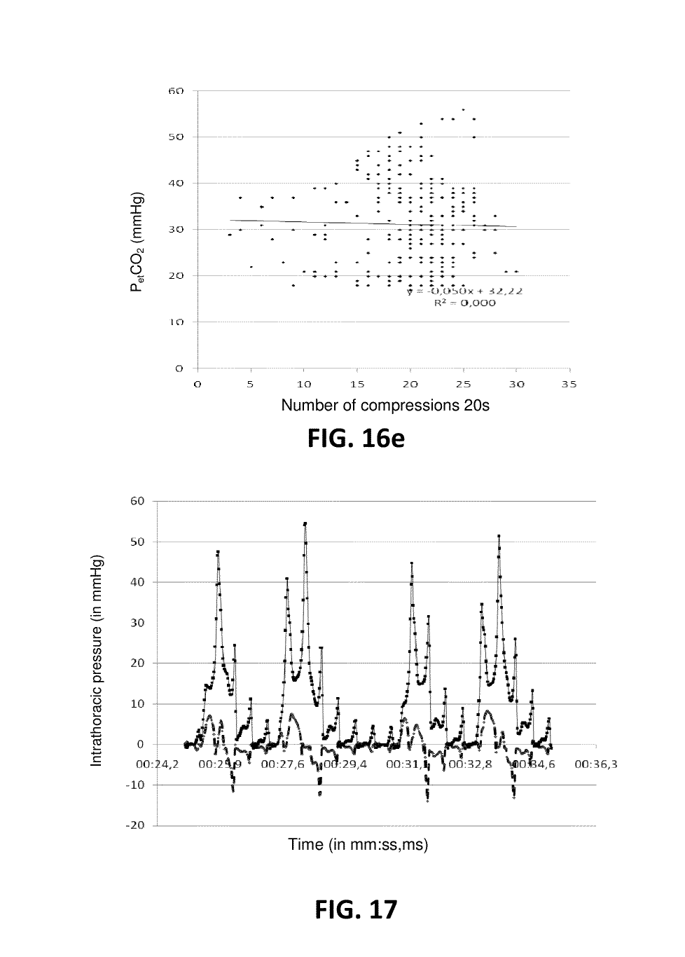

FIG. 16a to FIG. 16e illustrates a functional relationship between the end-tidal CO₂ and different parameters of the resuscitation for individual patients. FIG. 16a illustrates the end-tidal CO₂ (expressed in mm Hg) as function of the median compression depth, expressed in cm. FIG. 16b illustrates the end-tidal CO₂ as function of the median intrathoracic pressure difference upon chest compression ΔCP. FIG. 16c illustrates the end-tidal CO₂ as function of the area under the curve of the total intrathoracic pressure (ITP). FIG. 16d illustrates the end-tidal CO₂ as function of the ventilation pressure. FIG. 16e illustrates the end-tidal CO₂ as function of the number of compressions. It can be seen that these different values all can have an effect on the end-tidal CO₂ and thus on the effect obtained with the resuscitation. It is to be noticed that the effect of variation in some parameters may be patient specific, i.e. it may be larger for some patients than for others, again being an illustration that individual optimization as obtained using embodiments of the present invention is advantageous. In the examples shown, it can for example be seen that for the particular resuscitation, optimization of the median compression depth or the median intrathoracic pressure difference may result in a change of more than 30% of the end-tidal CO₂, while optimization of the number of compressions may result in a change of more than 10% of the end-tidal CO₂. Furthermore, these experimental results indicate that optimization of more than one parameter may be advantageous. The latter may be optimization one by one or optimization in group or simultaneously, as indicated above.

FIG. 17 illustrates the pressure difference ΔCP for the pressure sensed using a distal sensor indicative of the effect of resuscitation on the pressure differences occurring in the patient.

1.-14. (canceled)

15. A system for providing control signals for ventilating and/or compressing, respectively, comprising
an information receiving device that receives information of a resuscitation of an individual patient, the information being information regarding different values of either or both a chest compression parameter and a ventilation parameter as a function of a parameter indicative of blood circulation,
a processor programmed to evaluate the different values of either or both the chest compression parameter and the ventilation parameter as function of the parameter indicative of blood circulation and deriving based thereon a preferred value for either or both the ventilation parameter and the chest compression parameter, and
a control signal generator that generates control signals according to the derived preferred ventilation parameter value and chest compression parameter value.

16. The system for controlling according to claim 15, wherein the information receiving device is configured to receive different values of a ventilation parameter as function of a parameter indicative of blood circulation and the processor is configured to evaluate the different values of the ventilation parameter as a function of the parameter indicative of blood circulation.

17. The system according to claim 15, wherein the information receiving device is configured to provide different values of a ventilation parameter as a function of blood circulation corresponding with a range of ventilation volumes.

18. The system according to claim 15, wherein the information receiving device comprises a pressure sensor that is arranged to sense tracheal pressure.

19. A system according to claim 18, wherein the information receiving device or the processor comprises a calculator that calculates a parameter representative for either or both the pressure difference by chest compression and the ventilation volume, based on tracheal pressure values.

20. The system according to claim 15, wherein the information receiving device, the processor and the signal control generator comprise part of a feedback loop,
the system being configured for, starting from a given ventilation volume/pressure or pressure difference by chest compression respectively,
providing a control signal corresponding to another parameter value for a ventilation volume/pressure or a stronger/deeper chest compression,
receiving information regarding a parameter representative for the ventilation and/or compression as a function of a parameter indicative of blood circulation
evaluating either or both the ventilation parameter value and the compression parameter value as a function of the parameter indicative of blood circulation,
and repeating said providing, receiving and evaluating until a parameter value indicative of a predetermined level or optimum level of blood circulation has been reached.

21. The system according to claim 20, wherein the control signal generator is configured to select a control signal corresponding with at least one of the ventilation parameter value and the compression parameter value according to the predetermined level of or maximum level of blood circulation.

22. The system according to claim 15, wherein the information receiving device is configured to obtain end-tidal CO2 measurements.

23. The system according to claim 15, wherein the system comprises a ventilator or compressor respectively, the system thus being a ventilating system or compressing system.

24. A system according to claim 15, wherein the system is implemented as a computer program product that, when executed on a computer, provides control signals for ventilating or compressing.

25. A method for providing control signals for ventilating or compressing, respectively, comprising the steps:
receiving information of a resuscitation of an individual patient, the information being information regarding different values of either or both a chest compression parameter and a ventilation parameter as a function of a parameter indicative of blood circulation,
evaluating the different values of either or both the chest compression parameter and the ventilation parameter as a function of the parameter indicative of blood circulation and deriving based there on a preferred value for either or both the ventilation parameter and the chest compression parameter, and
generating control signals according to either or both the derived preferred ventilation parameter value and the chest compression parameter value for controlling ventilation and/or compression.

26. The method according to claim 25, including, starting from a given ventilation parameter or chest compression parameter,
providing a control signal corresponding to a different ventilation parameter value or a different chest compression parameter,
receiving information regarding a chest compression parameter or ventilation parameter as function of a parameter indicative of blood circulation,
evaluating either or both the ventilation parameter value and the compression parameter value as function of the parameter indicative of blood circulation and repeating said providing, receiving and evaluating until a parameter value indicative of a predetermined level or optimum level of blood circulation has been reached

27. A data carrier comprising a non-transient set of instructions that, when executed on a computer, perform a method that provides control signals for ventilating or compressing, respectively, the method comprising receiving information of a resuscitation of an individual patient, the information being information regarding different values of either or both a chest compression parameter and a ventilation parameter as a function of a parameter indicative of blood circulation, evaluating the different values of either or both the chest compression parameter and the ventilation parameter as a function of the parameter indicative of blood circulation and deriving based thereon a preferred value for either or both the ventilation parameter and the chest compression parameter, and generating control signals according to the derived preferred ventilation parameter value and/or chest compression parameter value for controlling ventilation and/or compression.

28. The data carrier according to claim 27, wherein the data carrier comprises a CD-ROM, a DVD, a flexible disk or floppy disk, a tape, a memory chip, a processor or a computer.
